(19) United States
(12) Patent Application Publication
(10) Pub. No.: US 2024/0001007 A1
(43) Pub. Date: Jan. 4, 2024

(54) Biomedical Implant Having Needle-Like Titania Nanorods

(71) Applicant: MEDOS INTERNATIONAL SARL, LE LOCLE (CH)

(72) Inventor: Thomas M DiMauro, Southboro, MA (US)

(73) Assignee: MEDOS INTERNATIONAL SARL, LE LOCLE (CH)

(21) Appl. No.: 17/860,724

(22) Filed: Jul. 8, 2022

Related U.S. Application Data
(63) Continuation-in-part of application No. 17/854,555, filed on Jun. 30, 2022.

(51) Int. Cl. A61L 31/08; A61B 17/70; A61L 31/02; A61L 31/16; C30B 29/32; C30B 7/14

(52) U.S. Cl. CPC A61L 31/088; A61B 17/7001; A61L 31/022; A61L 31/16; C30B 29/32; C30B 7/14; A61L 2300/404; A61L 2400/12; A61L 2420/02

(57) ABSTRACT

A biomedical implant having a surface having nanorods comprising at least 50% titania extending therefrom, wherein the nanorods are substantially needle-like having an average diameter of less than 50 nm, wherein the nanorods have an areal density of at least 25 nanorods/um², preferably obtained by contacting a biomedical implant having a surface comprising titanium with an acidic solution comprising ethylene glycol and a titania precursor capable of hydrolysis to titania for a time sufficient to epitaxially grow titania nanorods on the surface.

### PRIORITY DATA

This patent application is a continuation-in-part (CIP) of co-pending US non-provisional patent application U.S. Ser. No. 17/854,555, entitled “Biomedical Implant having Conical-Tipped Titania Nanorods”, (DiMauro), (MED6116USNP1), filed Jun. 30, 2022, the specification of which is incorporated by reference in its entirety.

### BACKGROUND OF THE INVENTION

Pedicle Screw loosening occurs in 1-15% of cases and in up to 60% of osteoporotic procedures. Yuan, Global Spine Journal, 1-8, 2021. Prinz reports that about 40% of cases of screw loosening showed a subclinical biofilm on the screw. Prinz, J. Neurosurg. Spine, 31, 424-9, 2019. Similarly, Agarwal reports that about 72% of cases of screw loosening showed a subclinical biofilm on the screw. Agarwal, Spine Surg Relat. Res., 2021, 5, 2, 104-8. Therefore, there is a desire to prevent bacterial infection-related biofilm development on pedicle screws.

The literature reports that implant surfaces that contain sharp points function to puncture and kill invading bacteria. For example Zhao, ACS Biomat Sci Eng., 2021 Jun. 14; 7(6):2268-2278 reports that one sharp surface displays good bactericidal activity against both Escherichia coli (98.6±1.23%) and Staphylococcus aureus (69.82±2.79%), which is attributed to a hybrid geometric nanostructure, i.e., the pyramidal structures of ˜23 nm in tip diameter formed with tall clustered wires, and the sharper sheets of ˜8 nm in thickness in-between these nanopyramids. Zhao explains that this nanostructure displays a unique mechano-bactericidal performance via the synergistic effect of capturing the bacteria cells and penetrating the cell membrane. Zhao further provides a good review of the “sharp” literature, reporting that previous attempts with using sharp-tipped implants provided bacterial kill rates of between about 40% and 90%.

The best bacterial kill rate reported thus far appears to be that of Ye, Bioactive Materials 15, 2022, 173-184, who developed alumina-coated ZnO rods. Ye reports that, driven by the equivalent adhesive force of S. aureus, the top-flat nanorods deform cell envelops, showing a bacteriostatic rate of 29% owing to proliferation-inhibited manner. The top-sharp nanorods puncture S. aureus, showing a bactericidal rate of 96% for the longer, and 98% for the shorter that simultaneously exhibits fair osseointegration in bacteria-infected rat tibias, identifying top sharpness as a predominate contributor to mechano-puncture activity. Thus, top-sharp nanorods are desirable for killing bacteria at an optimal rate. Although Ye demonstrates that nanorod tips forming a conical angle of less than about 138 degrees are useful for puncturing and killing bacteria, Ye actual conical angle that produced the high killing rate was only about 50 degrees. Therefore, it is clear that extremely sharp tips are best for killing bacteria. Because Ye worked with alumina-coated ZnO nanorods and it is believed that alumina-coated ZnO is not an FDA approved biomaterial, there remains a need for a technology that can produce a sharp tipped nanorods with an FDA approved biomaterial.

Sjostrom discloses a titanium substrate having titania nanorods with pyramidal-shaped tips (made with a process including bubbling 50 sccm acetone). Sjostrom, Materials Letters, 167, 22-26, 2016 at 25. However, FIG. 1a of Sjostrom discloses a tip density of such pyramids of only about 1-2 tips/um², a density that is very low.

### SUMMARY OF THE INVENTION

It has been noted by the present inventor that the literature has fairly consistently reported the production of sharp titania nanorods having conical tips by simply contacting a fluorine-doped tin oxide (FTO) substrate with a heated acidic solution comprising a titania precursor capable of hydrolysis to titanium dioxide. In particular, the literature has reported this technology in at least the following articles:
a) Wang, Phys. Chem. Chem. Phys., 2011, 13, 7008-13 (“Wang I”);
b) Harris-Lee, New J. Chem., 2022, 46, 8385;
c) Bade, SN Applied Sciences, 2019, 1, 1073;
d) Yan, ACS Appl. Mater. Interfaces, 2015, 7, 3791-6 and Yan, RSC Advances, 2015, 5, 95939;
e) Mbulanga, Ph.D. Thesis, “Development of Titanium Dioxide for Photo-Electrochemical Hydrogen Production”, 2019;
f) Gao, Chinese J. Chemical Physics., 30, 5, 2017, 576;
g) Prathan, Scientific Reports, 2020, 10, 8065;
h) Cao, Sensors and Actuators B, 156, 2011, 114-119;
i) Liu, J. Am. Chem. Soc., 2009, 131, 3985-90;
j) Navab, AIP Conf. Proc., 1920, 020015, 2018;
k) Xia, RSC Advances, 3, 39, 2013, 17668
l) Wang, Scientific Reports, 5, 9305, 2015 (“Wang II”)
   The commonality of technology within these articles is striking in that they all simply contacted a fluorine-doped tin oxide (FTO) substrate with a 150-180 degree C., 4-6 M HCl solution comprising a few percent titania precursor titanium butoxide for 2-24 hours. Over half of the articles disclose SEM photographs of the resulting nanorods as having conical termination tips, while the remainder disclose termination tips that are jagged and contain step-edges. Table I summarizes the approximate reaction conditions for these articles and their impressive results:

TABLE I

| | [Titanium | [HCl] | | Temp | Anneal | Tip | |
|---|---|---|---|---|---|---|---|
| Author | Butoxide]% | (M) | Time(hr) | (C.) | (C./hr/atm) | Description | FIG. |
| Wang | 1.6 | 6 | 20 | 150 | 450/0.5 | ~45° conical | 1c |
| Harris-Lee | 1.4 | 6 | 3 | 180 | 550/4/vac | ~60° Conical | 2C-E |
| Bade | 16 | 4 | 24 | 150 | 300-600 | ~60° Conical | 8 |
| Yan(2) | 1 | 6 | 2 | 180 | 450/2/air | ~45° Conical | 1b |
| Mbulanga | 1.6 | 6 | 20 | 150 | — | Step-edge jagged | 3-3b |
| Gao | 1.6 | 6 | 20 | 150 | — | Conical? | 2b |
| Prathan | 0.7-1 | 4 | 2 | 150 | — | ~90° Conical/rounded | 3k/8a, 8d |
| Cao | 2 | 6 | 4 | 150 | — | Step-edge | 2b |
| Liu | 1.6 | 6 | 20 | 150 | — | Step-edge | 2B |
| Navab | ? | 6 | 12 | 150 | — | ~30° Conical (12 hr) | 3c |
| | | | | | | Rounded (7 hr) | |
| Xia | 2.5 | 6 | 12 | 150 | — | ~90° Conical | S1-c |
| Wang II | 2.7 | 6 | 10 | 150 | — | Step-edge | 1 |

It is believed that the desirable sharp conical tip result reported in Table I can be replicated with titanium-based implants by simply replacing the FTO substrate reported in the literature with a titanium-based implant. Such a replacement will produce a novel titanium implant having conically tipped nanorods that may be as lethal as the Ye technology in killing undesirable bacteria.

There is reason to believe that such a replacement will lead to the desired result. In particular, there is reason to believe that titania can epitaxially grow on titanium dioxide substrates. For example, Biapo used TTIP as a titania precursor and grew titania nanorods on a substrate having a titanium dioxide seed layer. Biapo, ACS Appl Mat. Interfaces, 2019, 11, 38, 35122-31. Because titanium-based implants commonly have a native titania layer thereon, the requisite titania seed layer is present.

Further it is appreciated that the areal density of conical nanorods of the substrates in this Table I body of work can be much higher than that disclosed on Sjostrom. For example, Wang I in FIG. 1B discloses nanorod densities on the substrate of about 100 nanorods/um². In contrast, FIG. 1a Sjostrom discloses a tip density of its pyramids of only about 1-2 tips/um²

Therefore, in accordance with the present invention, there is provided a biomedical implant having a surface having nanorods comprising at least 50% titania extending therefrom, wherein the nanorods terminate in a substantially conical tip, wherein the nanorods have a density on the implant surface of at least 10 nanorods/um².

### DETAILED DESCRIPTION OF THE INVENTION

For the purposes of the present invention, nanorods with pyramidal-shaped tips are considered to have conical tips, as that is their appearance in an SEM side-view photo thereof.

Preferably, the bulk material of the implant comprises at least 50% titanium, and is more preferably Ti6Al4V, which comprises about 90% titanium. However, it may also be commercially pure titanium.

In some embodiments, there is provided a biomedical implant having a surface having nanorods consisting essentially of titania extending therefrom, wherein the nanorods terminate in a substantially conical tip.

Preferably, the conical tip forms a cone angle of no more than 90 degrees, more preferably no more than 60 degrees, most preferably no more than 45 degrees.

In some embodiments, the implant is a spinal implant, preferably a pedicle screw or an interbody fusion cage. In other embodiments, the implant is selected from the group consisting of a hip implant (such as an acetabular cup or a femoral insert); a knee implant (such as a tibial tray or a femoral stem), a shoulder implant and a trauma implant (such as a plate or a nail).

In some embodiments, the nanorods have an average mid-height diameter of between about 50 nm and 300 nm.

In some embodiments, the nanorods have an average length of between about 500 nm and about 3000 nm.

In some embodiments, the nanorods extend substantially in the same direction. That is, at least 85% of the nanorods extends extend at an angle of between 45 degree and 135 degrees from the implant surface.

Wang I discloses nanorod densities on the substrate of about 100 nanorods/um², respectively. Therefore, assuming the nanorods of Wang I can be replicated on titanium-based substrates, in some embodiments, the titania nanorods have a density on the implant of at least 10 nanorods/um², preferably at least 25 nanorods/um², more preferably at least 50 nanorods/um², most preferably at least 75 nanorods/um². It is believed that these greater densities will produce more effective bacterial killing rates than the lower pyramid-tipped densities of Sjostrom.

Although the implants having the conical tips are preferred, it is nonetheless believed that the jagged tips having step edges are also useful for killing bacteria. Therefore, in some embodiments, there is provided a biomedical implant having a surface having nanorods comprising titania extending therefrom, wherein the nanorods terminate in a substantially jagged tip having step edges. Preferably, the nanorods consist essentially of titania.

In some embodiments, there is provided a method of making a biomedical implant comprising the steps of:
a) contacting a biomedical implant having a surface comprising titanium with an acidic solution comprising a titania precursor capable of hydrolysis to titania for a time sufficient to epitaxially grow titania nanorods on the surface;
b) optionally, annealing the implant.

In some embodiments, the titania precursor is selected from the group consisting of titanium butoxide, titanium tetraisopropoxide (TTIP) and titanium tetrachloride, and is preferably titanium butoxide.

In one article involving the hydrothermal synthesis of titania with TTIP as the titania precursor, Yamazaki discloses alpha-hydroxy acids such as lactic acid and glycolic acid as “structure-directing agents”. Yamazaki, ACS Omega, 2021, 6, 31557-65. Therefore, in some embodiments, an alpha-hydroxy acid is added to the aqueous acidic solution comprising the titanium precursor as a way of directing formation of the conical tips.

It is noted that there are some commercial pedicle screws (e.g., Nanovis) having titania nanotubes, thereby demonstrating that titania nanostructures have the strength to withstand screw insertion. It is further noted that the literature on titania nanotubes generally reports SEM pictures showing the nanotubes closely packed together. Because the technology disclosed in Table I generally produces nanorods with a fair amount of spacing therebetween, the question is raised as to whether the nanorods of the present invention will have sufficient strength to withstand screw insertion. Therefore, in accordance with the present invention, there is provided a biomedical implant having titania nanorods extending therefrom, wherein the nanorods having a spacing therebetween, and wherein the spacing is filled with a coating. It is believed the coating will provide mechanical strength to the implant and protect the nanorods it envelops during screw insertion. Preferably, the coating is a resorbable polymeric coating, such as polyglycolic acid. In other embodiments, the coating comprises calcium phosphate such as hydroxyapatite.

### Prophetic Example I

This prophetic example reports a recipe for making the inventive biomedical implant and essentially adopts the technology disclosed in Wang I for making titania nanorods, but with a titanium alloy replacing FTO.

In particular, 12 mL of deionized water was mixed with 12 mL of concentrated hydrochloric acid (mass fraction 36.5-38%). The mixture was stirred under ambient conditions for 5 minutes before adding 0.4 mL of titanium butoxide (Beijing Chemical Co.). After stirring for another 5 minutes, the mixture was placed in a Teflon-lined stainless steel autoclave of 45 mL volume. Then, a Ti6Al4V spinal pedicle screw, ultrasonically cleaned for 60 minutes in a mixed solution of deionized water, acetone and 2-propanol (volume rations 1:1:1) was placed at an angle against the wall of the Teflon liner. The hydrothermal synthesis was conducted at 150° C. for 20 hours in an electric oven. After the synthesis, the screw as taken out, rinsed extensively in deionized water and dried in ambient air.

### Prophetic Example II

This prophetic example essentially follows Prophetic Example I above, but then further anneals the resulting nanorod-laden pedicle screw at 450° C. for 30 minutes.

### Second Aspect of the Invention

It has further been noted by the present inventor that the FIG. 5a microstructure disclosed in Biapo, supra, an article directed to chemical sensing, has many features desirable in an anti-infective biomedical implant. In particular, Biapo reports base conditions in which a silicon substrate is coated with titanium, and then the titanium is oxidized to titania, and then this titania-coated substrate is contacted in a base experiment with a liquid mixture of 0.25 ml TTIP, 15 ml HCl, 15 ml ethanol and 0.5 ml TEACL-saturated ethanol, at 150° C. for 8 hours, and a second experiment (FIG. 5a) replacing ethanol with ethylene glycol in the presence of HCl and 0.25 ml TTIP at 150° C. for 8 hours. Biapo reports that the resulting FIG. 5a nanorods are “needle-like”, have a diameter of only 13.5 nm, and are vertically aligned. FIG. 5a of Biapo further discloses a nanorod areal density of about 500 nanorods/um², and an average nanorod length of about 500 nm.

Because the FIG. 5a experiment of Biapo works with a titania-coated substrate, and titanium or titanium alloy implants also have a native titania coat or can be engineered to possess one, it is believed that the results of Biapo FIG. 5a can be translated directly onto that of a titanium-comprising biomedical implant. In particular, it is believed that following the Biapo FIG. 5a experiment by replacing the titanium-coated silicon substrate with a titanium-based biomedical implant will lead to substantially the same FIG. 5a microstructure on the implant as reported by Biapo.

Therefore, in accordance with a second aspect of the present invention, there is provided a biomedical implant having a surface having nanorods comprising at least 50% titania extending therefrom, wherein the nanorods are substantially needle-like having an average diameter of less than 15 nm. Preferably, the nanorods have an areal density of at least 25 nanorods/um²; more preferably at least 50 nanorods/um², more preferably at least 75 nanorods/um², more preferably at least 100 nanorods/um², more preferably at least 200 nanorods/um².

In some embodiments of this second aspect, the implant is a spinal implant, preferably a pedicle screw or an interbody fusion cage. In other embodiments, the implant is selected from the group consisting of a hip implant (such as an acetabular cup or a femoral insert); a knee implant (such as a tibial tray or a femoral stem), a shoulder implant and a trauma implant (such as a plate or a nail).

In some embodiments of the second aspect the nanorods have an average length of less than 1000 nanometers, preferably less than 750 nm, and the nanorods extend substantially in the same direction.

In another embodiment of the second aspect of the present invention, there is provided a biomedical implant having a surface having nanorods comprising at least 50% titania extending therefrom, wherein the nanorods are substantially needle-like having an average diameter of less than 50 nm, wherein the nanorods have an areal density of at least 25 nanorods/um². Preferably, the nanorods have an average diameter of less than 40 nm and an areal density of at least 50 nanorods/um²; more preferably, the nanorods have an average diameter of less than 30 nm and an areal density of at least 75 nanorods/um²; more preferably, the nanorods have an average diameter of less than 25 nm and areal density of at least 100 nanorods/um²; more preferably, the nanorods have an average diameter of less than 20 nm and areal density of at least 200 nanorods/um².

Preferably, the bulk material of the implant of this second aspect comprises at least 50% titanium, and is more preferably Ti6Al4V, which comprises about 90% titanium. However, it may also be commercially pure titanium. In some embodiments, the implant is oxidized in accordance with Biapo in order to obtain a titania overcoat.

### Prophetic Example III

This prophetic example carries out the teachings of Biapo with respect to its FIG. 5a, and simply replaces its titania-coated substrate with a titanium-based pedicle screw which has a crystallized oxide layer thereon.

First, the titanium-based pedicle screw is annealed in air flow at 800° C. for 6 hours (ramp of 10° C./min) to form a crystallized oxide layer.

As a base condition, 15 ml HCl (37%) and 15 ml ethanol are mixed and magnetically stirred for 5 minutes. Then, 0.25 ml TTIP and 0.5 ml ethanol saturated with triethylamine hydrochloride salt (TEACL) were added to the mixture. The obtained transparent solution was stirred for another 5 minutes and then transferred to a 100 ml Teflon-lined stainless autoclave. A piece of silicon-based substrate was attached to a sample holder so that the substrate was completely immersed in the precursor solution. The reaction was carried out at 150° C. for 8 hours.

In this prophetic example, the base condition is modified by replacing the silicon substrate with an annealed Ti pedicle screw, and replacing ethanol with ethylene glycol in the presence of HCl and 0.25 ml TTIP at 150° C. for 8 hours.

I claim:

1. A biomedical implant having a surface having nanorods comprising at least 50% titania extending therefrom, wherein the nanorods are substantially needle-like and have an average diameter of less than 15 nm.

2. The implant of claim 1 wherein the nanorods have an areal density of at least 25 nanorods/um².

3. The implant of claim 1 wherein the nanorods have an areal density of at least 50 nanorods/um².

4. The implant of claim 1 wherein the nanorods have an areal density of at least 75 nanorods/um².

5. The implant of claim 1 wherein the nanorods have an areal density of at least 100 nanorods/um².

6. The implant of claim 1 wherein the implant has a bulk comprising at least 50% titanium.

7. The implant of claim 1 wherein the implant is a spinal implant.

8. The implant of claim 1 wherein the implant is a pedicle screw.

9. The implant of claim 1 wherein the nanorods have an average length of less than 1000 nanometers

10. The implant of claim 1 wherein the nanorods extend substantially in the same direction.

11. A biomedical implant having a surface having nanorods comprising at least 50% titania extending therefrom, wherein the nanorods are substantially needle-like and have an average diameter of less than 50 nm, wherein the nanorods have an areal density of at least 25 nanorods/um².

12. The implant of claim 11 wherein the nanorods have an average diameter of less than 40 nm and an areal density of at least 50 nanorods/um².

13. The implant of claim 11 wherein the nanorods have an average diameter of less than 30 nm and an areal density of at least 75 nanorods/um².

14. The implant of claim 11 wherein the nanorods have an average diameter of less than 25 nm and areal density of at least 100 nanorods/um².

15. The implant of claim 11 wherein the implant has a bulk comprising at least 50% titanium.

16. The implant of claim 11 wherein the implant is a spinal implant.

17. The implant of claim 11 wherein the implant is a pedicle screw.

19. The implant of claim 11 wherein the nanorods have an average length of less than 1000 nanometers

20. The implant of claim 11 wherein the nanorods extend substantially in the same direction.

21. A method of making a biomedical implant comprising the steps of:
a) contacting a biomedical implant having a surface comprising titanium with an acidic solution comprising ethylene glycol and a titania precursor capable of hydrolysis to titania for a time sufficient to epitaxially grow titania nanorods on the surface.

22. The method of claim 21 wherein the titanium precursor is selected from the group consisting of titanium butoxide, TTIP and titanium tetrachloride.

23. The method of claim 21 wherein the titanium precursor is TTIP.
